(19) United States
(12) Patent Application Publication
(10) Pub. No.: US 2017/0247314 A1
(43) Pub. Date: Aug. 31, 2017

(54) FRAGRANCES FROM THE ESTERS OF FATTY ACIDS

(71) Applicant: P2 Science, Inc., Woodbridge, CT (US)

(72) Inventors: Patrick FOLEY, New Haven, CT (US); Yonghua YANG, New Haven, CT (US)

(21) Appl. No.: 15/501,739

(22) PCT Filed: Aug. 6, 2015
(86) PCT No.: PCT/US2015/044012

§ 371 Date: Feb. 3, 2017

Related U.S. Application Data
(60) Provisional application No. 62/034,037, filed on Aug. 6, 2014.

(51) Int. Cl. C07C 69/533; C07C 68/00; C07C 45/61; C07C 47/19; C07C 29/32; C07C 33/025

(52) U.S. Cl. CPC C07C 69/533; C07C 29/32; C07C 33/025; C07C 45/61; C07C 47/19; C07C 68/00

(57) ABSTRACT

The invention relates to the generation of compounds, e.g., fragrance molecules with desirable olfactory properties that can be derived from readily available fatty acids.

### RELATED APPLICATION

This application claims priority to, and the benefit of, U.S. Ser. No. 62/034,037, filed on Aug. 6, 2014, the contents of which are incorporated herein by reference in their entireties.

### BACKGROUND OF THE INVENTION

Esters of fatty acids can be used to make fragrances, as described herein.

New fragrances and means of obtaining them from readily available starting materials are desired.

### SUMMARY OF THE INVENTION

In one aspect, the invention features a compound according to Formula I, or a salt thereof,

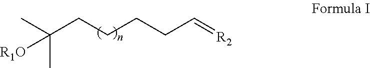

wherein:
R₁ is H, C₁₋₆ alkyl, or —C(O)C₁₋₆ alkyl;
R₂ is O, CH₂, or CHC₁₋₆ alkyl; and
n is an integer from 0 to 6.

In another aspect, the invention features a method of producing a compound of Formula I, or a salt thereof, wherein R₁ is H, C₁₋₆ alkyl, or —C(O)C₁₋₆ alkyl; R₂ is O, CH₂, or CHC₁₋₆ alkyl; and n is an integer from 0 to 6. The method comprises providing a compound of Formula III

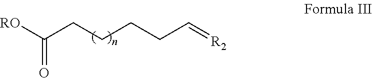

wherein R is C₁₋₆ alkyl or —CH₂CH(OR_{G})CH₂OR_{G}, wherein R_{G} is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and reacting the compound of Formula III with at least two equivalents of a methylating agent under conditions appropriate to obtain the compound of Formula I.

In another aspect, the invention features an alternate method of producing a compound of Formula I, or a salt thereof, wherein R₁ is H, C₁₋₆ alkyl, or —C(O)C₁₋₆ alkyl; R₂ is O, CH₂, or CHC₁₋₆ alkyl; and n is an integer from 0 to 6. The method comprises providing a compound of Formula IV

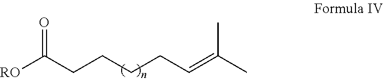

wherein R is H, C₁₋₆ alkyl, or —CH₂CH(OR_{G})CH₂OR_{G}, wherein R_{G} is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and reacting the compound of Formula IV with an acid followed by (i) etherifying the compound with a C₁₋₆ alcohol to produce compounds wherein R is C₁₋₆ alkyl or (ii) hydroxylating the compound with water to produce compounds wherein R is H; and converting the ester to the corresponding aldehyde.

In another aspect, the invention features a compound of Formula VI,

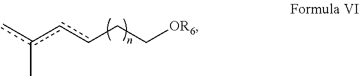

or a salt thereof, wherein:
R₆ is H, C₁₋₆ alkyl, or —C(O)C₁₋₆ alkyl;
n is and integer from 1 to 6; and
at least one of the [symbol] is a double bond, and the remaining [symbol] are single bonds, provided that two adjacent [symbol] are not both double bonds.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. In addition, the materials, methods, and examples are illustrative only and are not intended to be limiting.

Other features and advantages of the invention will be apparent from the following detailed description and claims.

### DETAILED DESCRIPTION OF THE INVENTION

This invention relates to the generation of novel fragrance molecules with desirable olfactory properties that can be derived from readily available starting materials, specifically fatty acids. The molecules represent new compositions of matter according to Formula I.

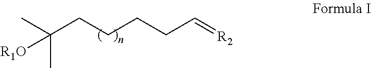

Examples of compounds of Formula I are shown below.

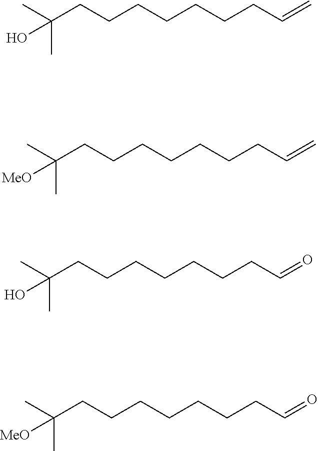

These molecules can be obtained from the esters of fatty acids such as oleic acid, decenoic acid, ricinoleic acid, linoleic acid, linolenic acid, and other unsaturated fatty acids. The invention relates to the addition of two equivalents of a methylating agent such as methyl lithium, methyl magnesium chloride, methyl magnesium bromide, or a functionally equivalent molecule, into the carbonyl of a fatty acid ester. The resulting fatty alcohol can then be further derivatized at the hydroxyl position, and/or at the unsaturated positions in the fatty acid using ozonolysis, metathesis, or both, either before or after addition of the methylating agent.

In one aspect the invention features a compound of Formula I:

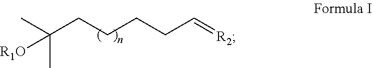

wherein:
R₁ is H, C₁₋₆ alkyl, or —C(O)C₁₋₆ alkyl;
R₂ is O, CH₂, or CHC₁₋₆ alkyl; and
n is an integer from 0 to 6.

In some embodiments, the compound is a compound according to Formula I.

In some embodiments, the compound is a compound according to Formula I wherein R₁ is H, CH₃, or CH₂CH₃.

In some embodiments, the compound is a compound according to Formula I wherein R₁ is —C(O)CH₃.

In some embodiments, the compound is a compound according to Formula I wherein R₂ is CH₂ or CHCH₃.

In some embodiments, the compound is a compound according to Formula I wherein R₂ is O.

In some embodiments, the compound is a compound according to Formula I, selected from:

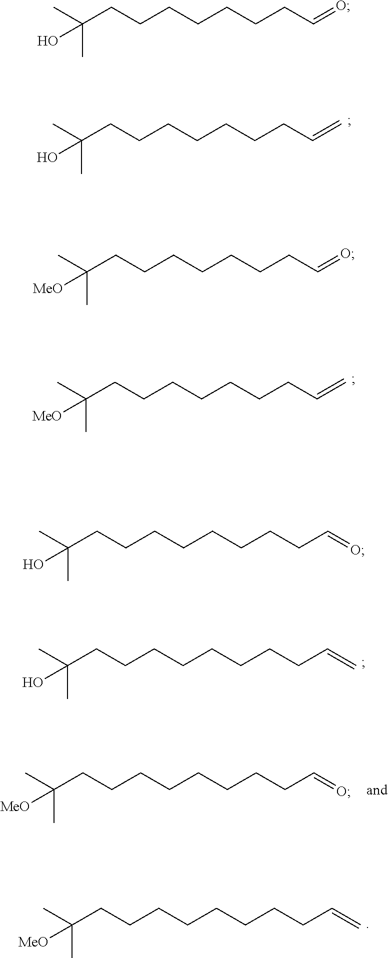

In another aspect, the invention features method of producing a compound of Formula I

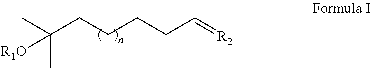

or a salt thereof, wherein R₁ is H, C₁₋₆ alkyl, or —C(O)C₁₋₆ alkyl; R₂ is O, CH₂, or CHC₁₋₆ alkyl; and n is an integer from 0 to 6; the method comprising:
providing a compound of Formula III

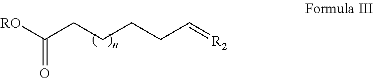

wherein R is C₁₋₆ alkyl or —CH₂CH(OR_{G})CH₂OR_{G}, wherein R_{G} is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and reacting the compound of Formula III with at least two equivalents of a methylating agent under conditions appropriate to obtain the compound of Formula I.

In some embodiments, the methylating agent in the method of producing a compound of Formula I is methyllithium, methylmagnesium chloride, or methylmagnesium bromide.

In some embodiments, the method of producing a compound of Formula I further comprises the step of performing reductive ozonolysis on the compound of Formula I wherein R₂ is CH₂ or CHC₁₋₁₀ alkyl to produce a corresponding compound of Formula I wherein R₂ is O.

In some embodiments, the method of producing a compound of Formula I further comprises the step of alkylating the compound of Formula I wherein R₁ is H with an alkylating agent to form a corresponding compound of Formula I wherein R₁ is C₁₋₆ alkyl.

In some embodiments, the method of producing a compound of Formula I further comprises the step of alkylating the compound of Formula I wherein R₁ is H with an alkylating agent to form a corresponding compound of Formula I wherein R₁ is C₁₋₆ alkyl.

In some embodiments, the method of producing a compound of Formulae I further comprises the step of performing reductive ozonolysis on the compound of Formula I wherein R₁ is C₁₋₆ alkyl and R₂ is CH₂ or CHC₁₋₁₀ alkyl to produce a corresponding compound of Formula I wherein R₂ is O.

In another aspect, the invention features a method of producing a compound of Formula I

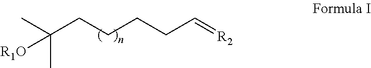

wherein R₁ is H, C₁₋₆ alkyl, or —C(O)C₁₋₆ alkyl; R₂ is O, CH₂, or CHC₁₋₆ alkyl; and n is an integer from 0 to 6; the method comprising:
providing a compound of Formula IV

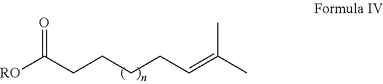

wherein R is H, C₁₋₆ alkyl or —CH₂CH(OR_{G})CH₂OR_{G}, wherein R_{G} is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and
reacting the compound of Formula IV with an acid followed by (i) etherifying the compound with a C₁₋₆ alcohol to produce compounds wherein R is C₁₋₆ alkyl or (ii) hydroxylating the compound with water to produce compounds wherein R is H; and converting the ester to the corresponding aldehyde.

In some embodiments, in the method of producing a compound of Formula I, the acid is H₂SO₄ or HCl.

In some embodiments, in the method of producing a compound of Formula I, the alcohol is methanol or ethanol.

In some embodiments, the method of producing a compound of Formula I further comprises the step of converting the C(O)OR group of the compound of Formula IV to CH₂OH, and optionally converting the CH₂OH group to a C(O)H group.

In some embodiments, the method of producing a compound of Formula I further comprises the step of converting the compound of Formula IV wherein RO is H to a compound of Formula IV wherein CH₂C(O)H is CH═CH₂.

In another aspect, the invention features a compound of Formula VI,

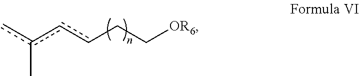

or a salt thereof,
wherein:
R₆ is H, C₁₋₆ alkyl, or —C(O)C₁₋₆ alkyl;
n is and integer from 1 to 6; and
at least one of the [symbol] is a double bond, and the remaining [symbol] are single bonds, provided that two adjacent [symbol] are not both double bonds.

In one iteration of the invention, an unsaturated fatty acid ester, such as an oleate or a 9-decenoate, can be derivatized with 2.0 equivalents of a nucleophilic methylating to generate an alcohol which can used as is or can be further derivatized. For example, this alcohol can then be cleaved with reductive ozonolysis at the unsaturated site to generate an aldehyde. This aldehyde can then be used as is or can be olefinated with a reagent such as a Wittig-type reagent to generate the desired olefin.

In another iteration of the invention, an unsaturated fatty acid ester, such as an oleate or a 9-decenoate, can be derivatized with 2.0 equivalents of a nucleophilic methylating to generate an alcohol which can used as is or can be further derivatized. For example, this alcohol can then be alkylated or acetylated at the hydroxyl position. This alkylated or acetylated product can be used as is, or can be taken on to reductive ozonolysis at the unsaturated site to generate an aldehyde. This aldehyde can then be used as is or can be olefinated with a reagent such as a Wittig-type reagent to generate the desired olefin.

Additional synthetic routes to these fragrance molecules can include starting with methyl azelaldehydate, which can be olefinated under standard conditions followed by dimethylation (Scheme 2). Alternatively, 10-methyl-9-undecenoic alkyl esters can be derived by metathesis with isobutylene or dimethyl butane and used as a starting material. This material can be etherified with a suitable alcohol or hydroxylated with water to give the desired functionality at one end of the molecule. For example, the olefin can be stirred overnight (e.g., from 0 to 100° C.; e.g., at 50° C.) in an organic solvent (e.g., methanol) with a lewis or bronsted acid present (e.g., methane sulfonic acid; e.g., 10% by wt.) to obtain the methoxy analog. Alternatively, water can be substituted for methanol to obtain the hydroxy analog.

The aldehyde can then be obtained through either selective reduction of the ester to the aldehyde, or by reduction of the ester to the alcohol, followed by oxidation to the aldehyde. The alcohol may also be isolated and characterized.

As shown below in Scheme 1, compounds of the invention can be prepared by a multi step process in which a compound of Formula III is alkylated with an alkylating agent to produce a compound of Formula I. The compound of Formula I can be converted to a corresponding aldehyde by performing reductive ozonolysis, or alternatively, it can be converted to the corresponding ether by performing alkylation or acetylation, and subsequently to a corresponding aldehyde by performing reductive onzonolysis.

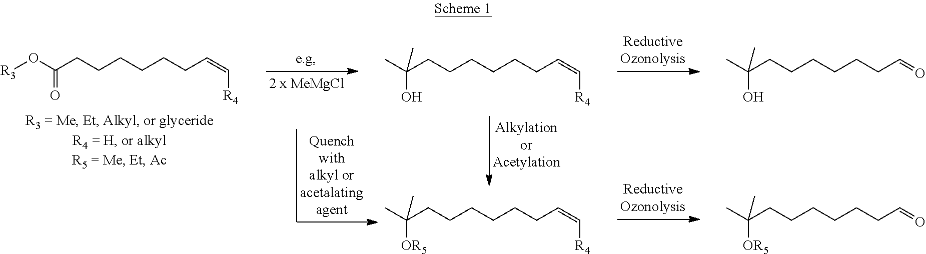

As shown below in Scheme 2, compounds of the invention can be prepared by a multi step process in which a compound of Formula III that contains an aldehyde group is converted to a corresponding olefin by performing an olefination step. The resulting olefin can subsequently be converted to a compound of Formula I by alkylating the ester group with an alkylating agent. The alcohol group of the resulting compound of Formula I can be converted to an ether by performing an additional alkylation or acetylation step.

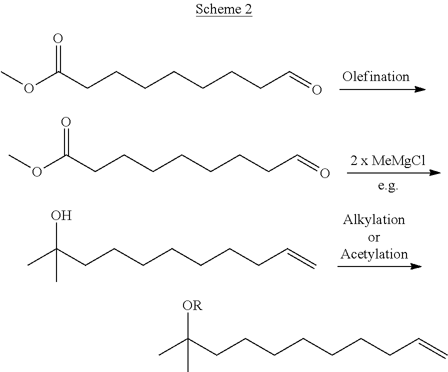

As shown below in Scheme 3, compounds of the invention can be prepared by a multi-step process in which the olefin group of a compound of Formula IV is converted to an ether by the addition of an alcohol in the presence of an acid. The resulting ether can subsequently be converted to a compound of Formula I by performing a reduction step to convert the ester group to an aldehyde.

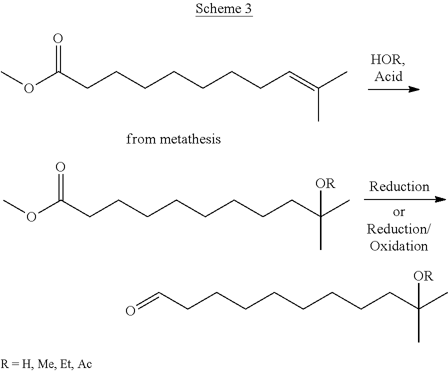

As shown below in Scheme 4, compounds of the invention can be prepared by either reduction or elimination procedures, starting with an ester or aldehyde of the invention. The reduction step shown may be accomplished using hydrogen gas and palladium, nickel, or copper, or alternatively, using a hydride such as aluminum hydride or borohydride. The elimination step shown may be accomplished using an acid.

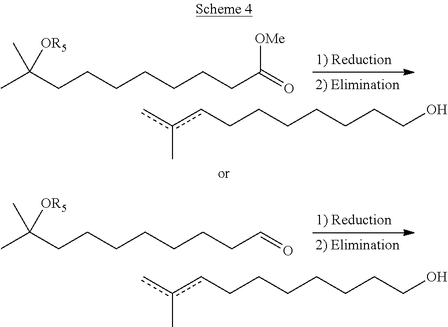

In one embodiment, under an inert gas, e.g., nitrogen, a solution of methylmagnesium bromide (e.g., in THF) is added, e.g., slowly, to a solution of methyl oleate (e.g., in THF), at a first temperature e.g., 0° C. (e.g., from −78 to 50° C). for e.g., 30 minutes (e.g., from 5 to 500 minutes). After stirring the mixture for e.g., 30 (e.g., from 5 to 500 minutes) minutes at e.g., 0° C. (e.g., from −78 to 50° C.), the mixture is stirred e.g., for 30 minutes (e.g., from 5 to 500 minutes), at a second temperature that is greater than the first temperature, e.g., room temperature (e.g., from −30 to 100° C). until all the starting material is consumed, e.g., as indicated by TLC. The mixture is then cooled down to, e.g., 0° C. (e.g., from −78 to 50° C). and quenched, e.g., with saturated ammonium chloride. All organic solvent (e.g., THF) is removed, e.g., by evaporation, and an acid e.g., acetic acid (e.g., 15% in water) is added to the mixture. The reaction mixture is then extracted with an organic solvent, e.g., ethyl acetate, and the organic solvent is then removed e.g., by evaporation to yield the crude fatty alcohol product.

In one embodiment, a mixture of fatty alcohol and water are cooled e.g., to 20° C., (e.g., from −5 to 60° C.) e.g., in a jacketed reactor, while stirring. A stream of O₃ e.g., in O₂, (e.g., 2-6% by weight) is diffused into the mixture e.g., at a flow rate of 10 L/min e.g., for 120 minutes (e.g., from 5 to 500 minutes). The reaction vessel is then purged with an inert gas (e.g., N₂) and the reaction mixture is transferred into a high-pressure reactor and charged with a catalyst e.g., palladium black. The reaction mixture is then stirred e.g., under a hydrogen atmosphere (e.g., at 350 psi) (e.g., from 5 to 500 psi) e.g., at 45-50° C., e.g., (e.g., from 0 to 100° C.) for 180 minutes (e.g., from 5 to 500 minutes) until all peroxide is consumed e.g., according to a titrated starch-iodine test. The reaction mixture is then cooled down and the catalyst is removed e.g., by filtration. The organic phase is then separated e.g., with a separatory funnel. Subsequently, the aqueous phase is extracted with an organic solvent e.g., ethyl acetate, and concentrated e.g., by solvent evaporation. The crude product is then washed e.g., with sodium carbonate (e.g., 10%), e.g., until the pH of the aqueous phase is approximately 8. The final product is then isolated e.g., by vacuum distillation (e.g., 2 in. wiped film, short-path distillation) and characterized.

In one embodiment, under an inert gas, e.g., nitrogen, potassium t-butoxide is added e.g., portion-wise, to a suspension of methyltriphenylphosphonium bromide e.g., in THF, e.g., at room temperature (e.g., from −78 to 60° C.), e.g., over the course of 10 minutes. The mixture is then stirred e.g., for 1 hour, e.g., at 50° C. (e.g., from −78 to 60° C.), and cooled down e.g., to 0° C. (e.g., from −78 to 50° C.), and methyl 9-oxononanoate is added e.g., in THF, e.g., slowly, e.g., by syringe, e.g., over 5 minutes (e.g., from 5 to 500 minutes). The cooling bath is removed and the reaction mixture is stirred e.g., for 2 hours (e.g., from 5 minutes to 500 minutes), e.g., at room temperature. Subsequently, ammonium chloride, e.g., as a saturated solution in water, is added to the mixture e.g., slowly, to quench the reaction. The aqueous and organic phases are then separated and the organic phase is set aside (first organic phase). The aqueous phase is then extracted with an organic solvent e.g., ethyl acetate, and all the organic phase (second organic phase) is combined with the first organic phase and concentrated e.g., by solvent evaporation. The final product is then isolated from the concentrated organic solution e.g., by column chromatography (e.g., silica gel, e.g., EtOAc/heptane, e.g., at 0-3%).

In one embodiment, under an inert gas, e.g., nitrogen, a solution of methylmagnesium bromide (e.g., in THF) is added, e.g., slowly, to a solution of methyl dec-9-enoate (e.g., in THF), at a first temperature e.g., 0° C. (e.g., from −78 to 60° C). for e.g., 5 minutes. After stirring the mixture e.g., for 30 minutes e.g., at 0° C., the mixture is stirred e.g., for 1.5 hours, at a second temperature that is greater than the first temperature, e.g., room temperature (e.g., from −78 to 70° C.) until all the starting material is consumed, e.g., as indicated by TLC. The mixture is then cooled down to, e.g., 0° C. (e.g., from −78 to 50° C). and quenched, e.g., with saturated ammonium chloride. All organic solvent (e.g., THF) is removed, e.g., by evaporation, and an acid e.g., acetic acid (e.g., 15% in water by vol.) is added to the mixture. The reaction mixture is then extracted with an organic solvent, e.g., ethyl acetate, and the solution is then concentrated e.g., by evaporation. The final product is then isolated from the concentrated organic solution e.g., by column chromatography (e.g., silica gel, e.g., EtOAc/heptane, e.g., at 3-7.5% by vol.).

Starting materials for the processes described herein include, but are not limited to, oleic acid, decenoic acid, ricinoleic acid, linoleic acid, and linolenic acid.

The details of one or more embodiments of the invention are set forth in the accompanying description below. Unless defined otherwise, all technical and scientific terms used herein have the same meaning as commonly understood by one of ordinary skill in the art to which this invention belongs. In the case of conflict, the present specification will control.

Unless otherwise indicated, it is to be understood that the terminology used herein is for the purpose of describing particular embodiments only and is not intended to be limiting. In this specification and in the claims that follow, reference will be made to a number of terms, which shall be defined to have the definitions set forth below.

As used herein, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, the phrase “having the formula” or “having the structure” is not intended to be limiting and is used in the same way that the term “comprising” is commonly used.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). In some formulae of the present application, one or more chiral centers are identified by an asterisk placed next to the chiral carbon. In other formulae, no chiral center is identified, but the chiral isomers are nonetheless covered by these formulae.

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Some compounds of the present invention can exist in a tautomeric form which is also intended to be encompassed within the scope of the present invention. “Tautomers” refers to compounds whose structures differ markedly in arrangement of atoms, but which exist in easy and rapid equilibrium. It is to be understood that the compounds of the invention may be depicted as different tautomers. It should also be understood that when compounds have tautomeric forms, all tautomeric forms are intended to be within the scope of the invention, and the naming of the compounds does not exclude any tautomeric form. Further, even though one tautomer may be described, the present invention includes all tautomers of the present compounds.

As used herein, the term “salt” can include acid addition salts including hydrochlorides, hydrobromides, phosphates, sulfates, hydrogen sulfates, alkylsulfonates, arylsulfonates, acetates, benzoates, citrates, maleates, fumarates, succinates, lactates, and tartrates; alkali metal cations such as Na⁺, K⁺, Li⁺, alkali earth metal salts such as Mg²⁺ or Ca²⁺, or organic amine salts, or organic phosphonium salts.

The term “alkyl” as used herein refers to a monovalent or bivalent, branched or unbranched saturated hydrocarbon group typically although not necessarily containing 1 to about 20 carbon atoms, such as methyl, ethyl, n-propyl, isopropyl, n-butyl, isobutyl, t-butyl, octyl, and the like.

The term “alkenyl” as used herein refers to a monovalent or bivalent, branched or unbranched, unsaturated hydrocarbon group typically although not necessarily containing 2 to about 20 carbon atoms and 1-10 carbon-carbon double bonds, such as ethylene, n-propylene, isopropylene, n-butylene, isobutylene, t-butylene, octylene, and the like.

The term “alkynyl” as used herein refers to a monovalent or bivalent, branched or unbranched, unsaturated hydrocarbon group typically although not necessarily containing 2 to about 20 carbon atoms and 1-10 carbon-carbon triple bonds, such as ethyne, propyne, butyne, pentyne, hexyne, heptyne, octyne, and the like.

By “substituted” as in “substituted alkyl,” “substituted alkenyl,” “substituted alkynyl,” and the like, it is meant that in the alkyl, alkenyl, alkynyl, or other moiety, at least one hydrogen atom bound to a carbon atom is replaced with one or more non-hydrogen substituents, e.g., by a functional group.

Examples of functional groups include, without limitation: halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (−(CO)−NH₂), mono-substituted C₁-C₂₄ alkylcarbamoyl (−(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂-OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O—)₂), phosphinato (—P(O)(O—)), phospho (—PO₂), phosphino (—PH₂), mono- and di-(C₁-C₂₄ alkyl)-substituted phosphino, mono- and di-(C₅-C₂₀ aryl)-substituted phosphino; and the hydrocarbyl moieties such as C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above.

In the present specification, the structural formula of the compound represents a certain isomer for convenience in some cases, but the present invention includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present invention.

### EXAMPLES

### Example 1

### Synthesis of (Z)-2-methylnonadec-10-en-2-ol

Under nitrogen, 208 mL of Methylmagnesium bromide solution (3M in THF from Sigma-Aldrich) was added slowly into methyl oleate (74 g, 0.25 mol) in THF (800 mL) at 0° C. over the course of 30 minutes. After stirring for 30 minutes at 0° C., the reaction was removed from the cooling bath and stirred for another 30 minutes. TLC showed all the starting material was consumed. The reaction was cooled down to 0° C. and quenched with saturated ammonium chloride. All the organic solvent (THF) was evaporated and 200 mL acetic acid (15% by vol. in water) was added into the mixture. The reaction mixture was extracted 2× with ethyl acetate (200 ml) and evaporation of the organic phase gave crude fatty alcohol product (90 g) that was taken on as is.

### (Z)-2-methylnonadec-10-en-2-ol

¹H NMR (CDCl₃, 400 MHz) δ 0.88 (t, J=7.2 Hz, 3H, —CH₃), 1.20 (s, 6H, —CH₃), 1.25-1.34 (m, 20H, —CH₂—), 1.43-1.47 (m, 2H, —CH₂—), 1.99-2.04 (m, 4H, —CH₂—), 5.29-5.41 (m, 2H, ═CH—).

### Example 2

### Synthesis of 9-hydroxy-9-methyldecanal

A mixture of fatty alcohol (85 g) and water (255 g) were cooled to 20° C. in a jacketed reactor while stirring. A 2-6% by weight stream of O₃ in O₂ was diffused into the mixture at a flow rate of 10 L/min for 120 minutes, while highest reaction temperature was 26° C. during the process. The reaction vessel was then purged with N₂ and the reaction mixture was transferred into a high-pressure reactor and charged with Palladium black (213 mg). The reaction mixture was stirred under hydrogen atmosphere (350 psi) at 45-50° C. for 180 minutes until all peroxide had been consumed according to a titrated starch-iodine test. The reaction mixture was then cooled down and filtered to remove the catalyst and the filtrate was placed in a separatory funnel. The organic phase was separated. The aqueous phase was extracted 2× with ethyl acetate (200 ml) and the orgaic phase was concentrated to remove solvent. The crude product was washed with sodium carbonate (10% by wt.) until the PH=8 of the aqueous phase. Vacuum distillation (2″ wiped film, short-path distillation) gave clean product 12.7 g.

### 9-hydroxy-9-methyldecanal

¹H NMR (CDCl₃, 500 MHz) δ 1.19 (d, J=1.0 Hz, 6H, —CH₃), 1.31-1.35 (m, 8H, —CH₂—), 1.42-1.46 (m, 2H, —CH₂—), 1.59-1.64 (m, 2H, —CH₂—), 2.39-2.43 (m, 2H, —CH₂—), 9.75-9.76 (m, 1H, —COH).

### Example 3

### Synthesis of methyl dec-9-enoate

Under nitrogen, potassium t-butoxide (13.1 g, 116 mmol) was added portion-wise into a suspension of methyltriphenylphosphonium bromide (41.6 g, 116 mmol) in THF (200 mL) at room temperature over the course of 10 minutes. The mixture was stirred for 1 hour at 50° C., and then cooled down to 0° C. and to add methyl 9-oxononanoate (10.8 g, 58 mmol) in THF (50 mL) slowly through syringe over 5 minutes. The cooling bath was removed and the reaction mixture was stirred for another 2 hours at room temperature. Saturated ammonium chloride solution (50 mL) was added slowly into the mixture to quench the reaction. Phases separated and the organic phase was collected. The aqueous phase was extracted 2× with ethyl acetate (200 ml) and all the organic phases were combined and concentrated to remove solvent. Column chromatograph gave 4.8 g of methyl dec-9-enoate in good purity (silica gel, EtOAc/heptane: 0-3% by vol.).

### methyl dec-9-enoate

¹H NMR (CDCl₃, 500 MHz) δ 1.25-1.40 (m, 8H, —CH₂), 1.59-1.65 (m, 2H, CH₂), 2.01-2.05 (m, 2H, —CH₂—), 2.30 (t, J=7.5 Hz, 2H, —CH₂—), 3.66 (s, 3H, —OCH₃), 4.91-5.00 (m, 2H, —CH₂—), 5.76-5.83 (m, 2H, ═CH2).

### Example 4

### Synthesis of 2-methylundec-10-en-2-ol

Under nitrogen, 14mL of Methylmagnesium bromide solution (3M in THF from Sigma-Aldrich) was added slowly into methyl dec-9-enoate (3.1 g, 16.8 mmol) in THF (50 mL) at 0° C. during 5 minutes. After stirring for 30 minutes at 0° C., removed the cooling bath and stirred for another 1.5 hours. TLC showed all the starting materials were consumed. Cooled down the reaction to 0° C. and quenched with saturated ammonium chloride. Evaporated all the organic solvent (THF) and added 40 mL acetic acid (15% in water by vol.) into the mixture. The aqueous layer (150 mL+50 mL) was extracted with ethyl acetate 2× (150 ml, then 50 ml) and all the organic phases were combined and concentrated to remove solvent. 1.1g of 2-methylundec-10-en-2-ol was then obtained following column chromatography in good purity (silica gel, EtOAc/heptane: 3-7.5% by vol.).

### 2-methylundec-10-en-2-ol

¹H NMR (CDCl₃, 500 MHz) δ 1.25 (s, 6H, —CH₃), 1.30-1.41 (m, 10H, CH₂), 1.43-1.48 (m, 2H, CH₂), 2.01-2.07 (m, 2H, —CH₂—), 4.91-5.02 (m, 2H, ═CH₂), 5.76-5.86 (m, 1H, ═CH—).

### INCORPORATION BY REFERENCE

The entire disclosure of each of the patent documents and scientific articles referred to herein is incorporated by reference for all purposes.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound of the Formula I: 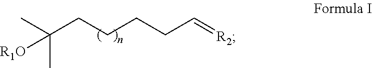
wherein:
R₁ is H, C₁₋₆ alkyl, or —C(O)C1-6 alkyl;
R₂ is O, CH₂, or CHC₁₋₆ alkyl; and
n is an integer from 0 to 6.

2. The compound of claim 1, wherein R₁ is H, CH₃, or CH₂CH₃.

3. The compound of claim 1, wherein R₁ is —C(O)CH₃.

4. The compound of claim 1, wherein R₂ is CH₂ or CHCH₃.

5. The compound of claim 1, wherein R₂ is O.

6. The compound of claim 1, wherein the compound is selected from the group consisting of:

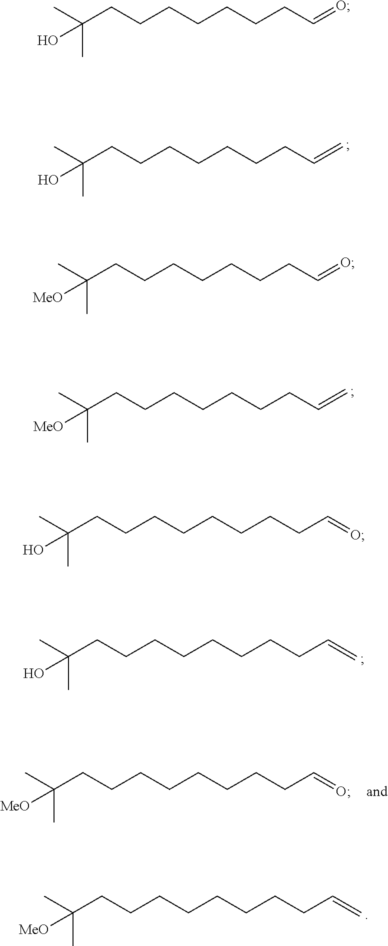

7. A method of producing a compound of claim 1: 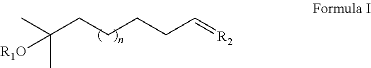
or a salt thereof, wherein R₁ is H, C₁₋₆ alkyl, or —C(O)C₁₋₆ alkyl; R₂ is O, CH₂, or CHC₁₋₆ alkyl; and n is an integer from 0 to 6; the method comprising:
providing a compound of Formula III 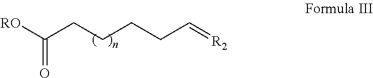
wherein R is C₁₋₆ alkyl or —CH₂CH(ORG)CH₂ORG, wherein RG is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and
reacting the compound of Formula III with at least two equivalents of a methylating agent under conditions appropriate to obtain the compound of Formula I.

8. The method of claim 7, wherein the methylating agent is methyllithium, methylmagnesium chloride, or methylmagnesium bromide.

9. The method of claim 7, further comprising the step of performing reductive ozonolysis on the compound of Formula I wherein R₂ is CH₂ or CHC₁₋₁₀ alkyl to produce a corresponding compound of Formula I wherein R₂ is O.

10. The method of claim 7, further comprising the step of alkylating the compound of Formula I wherein R₁ is H with an alkylating agent to form a corresponding compound of Formula I wherein R₁ is C1-6 alkyl.

11. The method of claim 10, further comprising the step of performing reductive ozonolysis on the compound of Formula I wherein R₁ is C₁₋₆ alkyl and R₂ is CH₂ or CHC₁₋₁₀ alkyl to produce a corresponding compound of Formula I wherein R₂ is O.

12. A method of producing a compound of claim 1: 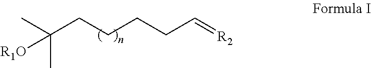
wherein R₁ is H, C₁₋₆ alkyl, or —C(O)C₁₋₆ alkyl; R₂ is O, CH₂, or CHC₁₋₆ alkyl; and n is an integer from 0 to 6; the method comprising:
providing a compound of Formula IV 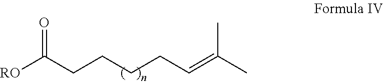
wherein R is H, C₁₋₆ alkyl or —CH₂CH(ORG)CH₂ORG, wherein RG is independently selected from the group consisting of hydrogen, —C(O)C₂₋₂₀ alkyl, and —C(O)C₂₋₂₀ alkenyl, R₂ is —CH₂ or —CHC₁₋₁₀ alkyl, and n is an integer from 0 to 6; and
reacting the compound of Formula IV with an acid followed by (i) etherifying the compound with a C₁₋₆ alcohol to produce compounds wherein R is C₁₋₆ alkyl or (ii) hydroxylating the compound with water to produce compounds wherein R is H; and
converting the ester to the corresponding aldehyde.

13. The method of claim 12, wherein the acid is H₂SO₄ or HCl.

14. The method of claim 12, wherein the alcohol is methanol or ethanol.

15. The method of claim 12, further comprising the step of converting the C(O)OR group of the compound of Formula IV to CH₂OH, and optionally converting the CH₂OH group to a C(O)H group.

16. The method of claim 15, further comprising the step of converting the compound of Formula IV wherein RO is H to a compound of Formula IV wherein CH₂C(O)H is CH═CH₂.

17. The compound of claim 1, wherein R₁ is H.

18. The compound of claim 1, wherein R₁ is CH3.

19. The compound of claim 1, wherein R₂ is CH2.

20. The compound of claim 1, wherein n is 4 or 5.
